(19) United States
(12) Patent Application Publication
(10) Pub. No.: US 2025/0073092 A1
(43) Pub. Date: Mar. 6, 2025

(54) DIAPER-BASED ANOMALY ANALYSIS AND PREWARNING METHOD, ELECTRONIC TAG COMPONENT, DIAPER, AND MONITORING DEVICE

(71) Applicant: Shenzhen Xingweixian Technology Co., Ltd, Shenzhen (CN)

(72) Inventors: Liankun Lai, Shenzhen (CN); Aixue Shu, Shenzhen (CN)

(21) Appl. No.: 18/825,215

(22) Filed: Sep. 5, 2024

(30) Foreign Application Priority Data
Sep. 5, 2023 (CN) 202311134370.X
Sep. 13, 2023 (CN) 202311177677.8
May 11, 2024 (CN) 202410582708.6

(51) Int. Cl. A61F 13/42; H01Q 1/22; H01Q 7/00; H01Q 9/20

(52) U.S. Cl. CPC A61F 13/42; H01Q 1/2225; H01Q 7/00; H01Q 9/20; A61F 2013/424

(57) ABSTRACT

The present disclosure discloses a diaper-based anomaly analysis and prewarning method, an electronic tag component, a diaper, and a monitoring device. Multi-algorithm analysis can be performed according to a first radio frequency signal sent by a first radio frequency electronic tag, a detected result from a human perception unit, and/or a second radio frequency signal sent by a second radio frequency electronic tag, so that an abnormal state of the liquid during the use of a diaper is accurately analyzed and prewarned, the inertial research and development thought of the skilled in the art continuing a research and development path in a physical direction of the diaper itself is broken, and a research and development path and a technical solution which are environment-friendly, safe in use, convenient to operate, controllable in cost and accurate in anomaly prewarning are successfully and non-obviously applied.

### CROSS REFERENCE TO RELATED APPLICATIONS

The present application claims the benefit of Chinese Patent Application Nos. 202311134370.X filed on Sep. 5, 2023, 202311177677.8 filed on Sep. 13, 2023 and 202410582708.6 filed on May 11, 2024. All the above are hereby incorporated by reference in their entirety.

### TECHNICAL FIELD

The present disclosure relates to the technical field of internet of things control, in particular to a diaper-based anomaly analysis and prewarning method, an electronic tag component, a diaper, and a monitoring device.

### BACKGROUND

It is well known that a diaper is a disposable nursing product which is mainly used for absorbing and locking urine to keep the skin of a wearer dry and sanitary, thereby achieving an important use value for infants and special crowds. However, an existing diaper does not have a function of actively reminding anomalies (for example, there is a situation that a diaper having absorbed and locked urine needs to be replaced, but is not replaced for a long time), which leads to a plurality of problems, such as bedsores, eczema and even decay on the skin, on the body of a user due to the untimely replacement of the diaper, thereby seriously affecting the health of the user.

At present, in order to solve this problem, the inertial research and development thought of research teams of a plurality of enterprises and professional research institutions is to perform continuous technical update in a physical direction of the diaper itself, such as a urine adsorption structure, a urine locking structure, a material permeability characteristic, and a structure so as to relieve a plurality of health problems caused by the untimely replacement of the diaper as much as possible. However, such continuous technical update in the physical direction can only relieve related use problems, but cannot avoid the continuous generation of the related use problems.

Therefore, how to break the inertial research and development thought of continuing a research and development path in a physical direction of the diaper itself so as to seek a research and development path and a technical solution which are safe in use, convenient to operate and controllable in cost in addition to the research and development path in the physical direction of the diaper itself has become an urgent technical problem to be solved.

### SUMMARY

In view of above reasons, it is necessary to provide a diaper-based anomaly analysis and prewarning method, an electronic tag component, a diaper, and a monitoring device so as to break the inertial research and development thought of the skilled in the art continuing a research and development path in a physical direction of a diaper itself so as to seek a research and development path and a technical solution which are environment-friendly, safe in use, convenient to operate, controllable in cost and accurate in anomaly prewarning in addition to the research and development path in the physical direction of the diaper itself.

In order to achieve the above-mentioned object, the present disclosure provides a diaper-based anomaly analysis method, the method being applicable to a diaper configured with an electronic tag component, and the electronic tag component including a first radio frequency electronic tag for detecting a first radio frequency signal representing that the diaper is located in a perceptible area and a second radio frequency electronic tag for detecting a second radio frequency signal representing the existence of a liquid, wherein the method includes a step of analyzing anomaly prewarning and a step of sending anomaly prewarning information, wherein
the step of analyzing anomaly prewarning includes:
sending a radio frequency signal in real time or regularly by a monitoring device so that the first radio frequency electronic tag of the electronic tag component sends the first radio frequency signal when generating electromagnetic induction with the radio frequency signal and the second radio frequency electronic tag sends the second radio frequency signal when generating electromagnetic induction with the radio frequency signal, detecting the first radio frequency signal in real time or regularly or after receiving a detection instruction sent by a user, analyzing a signal value of the detected first radio frequency signal based on a first analysis algorithm so as to analyze whether the second radio frequency signal needs to be analyzed, and if the second radio frequency signal needs to be analyzed, detecting the second radio frequency signal in real time or regularly, and analyzing a signal value of the detected second radio frequency signal based on a second analysis algorithm so as to analyze whether anomaly prewarning information needs to be sent; or
sending a radio frequency signal in real time or regularly by a monitoring device so that the first radio frequency electronic tag of the electronic tag component sends the first radio frequency signal when generating electromagnetic induction with the radio frequency signal and the second radio frequency electronic tag sends the second radio frequency signal when generating electromagnetic induction with the radio frequency signal, detecting the first radio frequency signal and the second radio frequency signal in real time or regularly or after receiving a detection instruction sent by a user, analyzing a signal value of the detected first radio frequency signal based on a first analysis algorithm so as to analyze whether the second radio frequency signal needs to be analyzed, and if the second radio frequency signal needs to be analyzed, analyzing a signal value of the detected second radio frequency signal based on a second analysis algorithm so as to analyze whether anomaly prewarning information needs to be sent; and
the step of sending anomaly prewarning information includes:
if anomaly prewarning information needs to be sent, sending anomaly prewarning information in a preset format to a predetermined electronic device by a monitoring device; or
if anomaly prewarning information needs to be sent, sending a prewarning information sending request to a background server by a monitoring device so that the background server sends anomaly prewarning information in a preset format to a portable electronic device in response to the sending request.

In order to achieve the above-mentioned object, the present disclosure further provides a diaper-based anomaly analysis method, the method is applicable to a diaper configured with a second radio frequency electronic tag, the second radio frequency electronic tag is used for detecting a second radio frequency signal representing the existence of a liquid, the method includes:
sending a radio frequency signal in real time or regularly by a monitoring device so that the second radio frequency electronic tag sends the second radio frequency signal when generating electromagnetic induction with the radio frequency signal;
detecting, by the monitoring device, the second radio frequency signal in real time or regularly or after receiving a detection instruction sent by a user, and analyzing a signal value of the detected second radio frequency signal based on a second analysis algorithm so as to analyze whether anomaly prewarning information needs to be sent; and
if the anomaly prewarning information needs to be sent, directly sending anomaly prewarning information in a preset format to a portable electronic device by the monitoring device; or if the anomaly prewarning information needs to be sent, sending a prewarning information sending request to a background server by the monitoring device so that the background server sends anomaly prewarning information in a preset format to a portable electronic device in response to the sending request.

In order to achieve the above-mentioned object, the present disclosure further provides a diaper-based anomaly analysis method, the method is applicable to a diaper configured with a second radio frequency electronic tag, the second radio frequency electronic tag is used for detecting a second radio frequency signal representing the existence of a liquid, and the method includes a step of detecting the second radio frequency signal, a step of analyzing the second radio frequency signal, and a step of sending anomaly prewarning information, wherein
the step of detecting the second radio frequency signal includes:
receiving and making a response to, by a monitoring device, an analysis instruction sent by an electronic device with a human perception unit, sending a radio frequency signal in real time or regularly so that the second radio frequency electronic tag sends the second radio frequency signal when generating electromagnetic induction with the radio frequency signal, and detecting the second radio frequency signal in real time or regularly; or
sending a radio frequency signal in real time or regularly by a monitoring device so that the second radio frequency electronic tag sends the second radio frequency signal when generating electromagnetic induction with the radio frequency signal, receiving and making a response to an analysis instruction sent by an electronic device with a human perception unit, and detecting the second radio frequency signal in real time or regularly;
the step of analyzing the second radio frequency signal includes:
analyzing, by a monitoring device, a signal value of the detected second radio frequency signal based on a second analysis algorithm so as to analyze whether anomaly prewarning information needs to be sent; and
the step of sending anomaly prewarning information includes:
if the anomaly prewarning information needs to be sent, directly sending anomaly prewarning information in a preset format to a portable electronic device by a monitoring device; or
if the anomaly prewarning information needs to be sent, sending a prewarning information sending request to a background server by a monitoring device so that the background server sends anomaly prewarning information in a preset format to a portable electronic device in response to the sending request.

In order to achieve the above-mentioned object, the present disclosure further provides a diaper-based anomaly analysis method, the method is applicable to a diaper configured with a second radio frequency electronic tag, the second radio frequency electronic tag is used for detecting a second radio frequency signal representing the existence of a liquid, and the method includes a step of detecting the second radio frequency signal, a step of analyzing the second radio frequency signal, and a step of sending anomaly prewarning information, wherein
the step of detecting the second radio frequency signal includes:
detecting, by a monitoring device, human information in a specific area based on a human perception unit in real time or regularly or after receiving a detection instruction sent by a user, analyzing the detected human information based on a third analysis algorithm so as to analyze whether the second radio frequency signal needs to be analyzed, if the second radio frequency signal needs to be analyzed, sending a radio frequency signal in real time or regularly so that an electric small loop antenna and half-wave dipole antennae in the second radio frequency electronic tag send the second radio frequency signal when generating electromagnetic induction with the radio frequency signal, and detecting the second radio frequency signal in real time or regularly; or
sending a radio frequency signal in real time or regularly by a monitoring device so that the second radio frequency electronic tag sends the second radio frequency signal when generating electromagnetic induction with the radio frequency signal, detecting human information in a specific area based on a human perception unit in real time or regularly or after receiving a detection instruction sent by a user, analyzing the detected human information based on a third analysis algorithm so as to analyze whether the second radio frequency signal needs to be analyzed, and if the second radio frequency signal needs to be analyzed, detecting the second radio frequency signal in real time or regularly;
the step of analyzing the second radio frequency signal includes:
analyzing, by a monitoring device, a signal value of the detected second radio frequency signal based on a second analysis algorithm so as to analyze whether anomaly prewarning information needs to be sent; and
the step of sending anomaly prewarning information includes:
if the anomaly prewarning information needs to be sent, directly sending anomaly prewarning information in a preset format to a portable electronic device by a monitoring device; or
if the anomaly prewarning information needs to be sent, sending a prewarning information sending request to a background server by a monitoring device so that the background server sends anomaly prewarning information in a preset format to a portable electronic device in response to the sending request.

In order to achieve the above-mentioned object, the present disclosure further provides an electronic tag component applicable to the above-mentioned diaper-based anomaly analysis method, and the electronic tag component includes a first radio frequency electronic tag for detecting a first radio frequency signal representing that the diaper is located in a perceptible area and a second radio frequency electronic tag for detecting a second radio frequency signal representing the existence of a liquid, wherein
the first radio frequency electronic tag includes a first conductive substrate, and the first conductive substrate includes a first substrate segment, a second substrate segment, and a transition substrate segment electrically connected to the first substrate segment and the second substrate segment;
the second radio frequency electronic tag includes a first half-wave dipole antenna, a second half-wave dipole antenna, and a transition antenna segment electrically connected to the first half-wave dipole antenna and the second half-wave dipole antenna;
the first substrate segment is provided with a linear slot and a first tag chip disposed on one side of the slot, the first substrate segment and the second substrate segment are horizontally misaligned in a length direction, and the second substrate segment is located on an upper side of the first substrate segment in a width direction;
the transition antenna segment is provided with an electric small loop antenna, the electric small loop antenna is provided with a second tag chip, and the first half-wave dipole antenna, the second half-wave dipole antenna and the transition antenna segment are located in the same straight line in the length direction; and
the second substrate segment is located on an upper side of the first half-wave dipole antenna, the second substrate segment and the first half-wave dipole antenna are parallel in the length direction, and the first substrate segment and the first half-wave dipole antenna are located in the same straight line in the length direction.

In order to achieve the above-mentioned object, the present disclosure further provides a diaper including a surface layer, an acquisition distribution layer, a water absorption layer, and a back sheet, and the diaper further includes the above-mentioned electronic tag component fixedly mounted on the acquisition distribution layer and/or the back sheet.

In order to achieve the above-mentioned object, the present disclosure further provides a monitoring device, the monitoring device includes a communication unit, a memory, an RFID card reader, and a processor in communication connection to the communication unit, the memory and the RFID card reader, respectively, the memory stores a diaper-based anomaly analysis and prewarning program callable and executable for the processor, and when the diaper-based anomaly analysis and prewarning program is called and executed by the processor, the above-mentioned diaper-based anomaly analysis method is implemented.

In order to achieve the above-mentioned object, the present disclosure further provides a nonvolatile computer-readable storage medium in which a diaper-based anomaly analysis and prewarning program callable and executable for at least one processor is stored, and when the diaper-based anomaly analysis and prewarning program is called and executed by the at least one processor, the above-mentioned diaper-based anomaly analysis method is implemented.

In the present disclosure, multi-algorithm analysis is performed according to a first radio frequency signal which is sent by a first radio frequency electronic tag mounted on a diaper and represents that the diaper is located in a perceptible area, a detected result from a human perception unit that the diaper is located in the perceptible area, and/or a second radio frequency signal which is sent by a second radio frequency electronic tag mounted on the diaper and represents the existence of a liquid, so that an abnormal state of the liquid during the use of the diaper is accurately analyzed and prewarned, the inertial research and development thought of the skilled in the art continuing a research and development path in a physical direction of the diaper itself is broken, and a research and development path and a technical solution which are environment-friendly, safe in use, convenient to operate, controllable in cost and accurate in anomaly prewarning are successfully and non-obviously applied in addition to the research and development path in the physical direction of the diaper itself.

### BRIEF DESCRIPTION OF THE ACCOMPANYING DRAWINGS

FIG. 1 is a schematic diagram of signal interaction of a diaper-based anomaly analysis and prewarning system provided in an embodiment of the present disclosure;

FIG. 2 is a schematic structural diagram of an electronic tag component 6 which can be mounted on a diaper 2 shown in FIG. 1 in an embodiment of the present disclosure;

FIG. 3 is a schematic diagram of a single structure of a first radio frequency electronic tag in the electronic tag component shown in FIG. 2;

FIG. 4 is a schematic diagram of a single structure of a second radio frequency electronic tag in the electronic tag component shown in FIG. 2;

FIG. 5 is a schematic diagram of a layered structure of the diaper 2 shown in FIG. 1;

FIG. 6 is a schematic diagram of a placement position of a monitoring device 1 shown in FIG. 1;

FIG. 7 is a schematic diagram of an internal structure of the monitoring device 1 shown in FIG. 1;

FIG. 8 is a schematic diagram of an internal structure of an RFID card reader 15 shown in FIG. 5;

FIG. 9 is a schematic flow diagram of a diaper-based anomaly analysis and prewarning method provided in a first embodiment of the present disclosure;

FIG. 10 is a schematic flow diagram of a diaper-based anomaly analysis and prewarning method provided in a second embodiment of the present disclosure;

FIG. 11 is a schematic flow diagram of a diaper-based anomaly analysis and prewarning method provided in a third embodiment of the present disclosure;

FIG. 12 is a schematic flow diagram of a diaper-based anomaly analysis and prewarning method provided in a fourth embodiment of the present disclosure; and

FIG. 13 is a schematic flow diagram of a diaper-based anomaly analysis and prewarning method provided in a fifth embodiment of the present disclosure.

The achievement of the purpose as well as functional characteristics and advantages of the present disclosure will be further exemplarily described in conjunction with embodiments and with reference to accompanying drawings.

### DETAILED DESCRIPTION OF ILLUSTRATED EMBODIMENTS

The principle and spirit of the present disclosure will be described below with reference to a plurality of specific embodiments. It should be understood that specific embodiments described herein are only intended to explain the present disclosure, rather than to limit the present disclosure.

It should be understood that “a plurality of” mentioned in the present application refers to two or more. In the description of the present application, unless otherwise noted, “/” denotes or, for example, A/B may denote A or B; the term “and/or” described herein only describes an association relationship among associated objects and denotes that three relationships may exist, for example, A and/or B may denote three situations that A exists alone; A and B exist at the same time; and B exists alone. In addition, in order to facilitate describing the technical solutions of the present application clearly, the same or similar items with the basically same function and effect are distinguished by adopting words, such as “first” and “second”. It can be understood by the skilled in the art that the words, such as “first” and “second”, do not limit an amount and an execution order, and are not limited to be certainly different.

Statements, such as “an embodiment” or “some embodiments”, described in the present application mean that specific features, structures or characteristics described in one or more embodiments of the present application are included in the embodiment. Therefore, statements, such as “in an embodiment”, “in some embodiments”, “in other embodiments”, and “in further embodiments”, appearing on different parts of the present application do not necessarily refer to the same embodiment, but mean “one or more of, rather than all embodiments, unless specifically emphasized otherwise. In addition, terms “including”, “include”, “being provided with” and variations thereof mean “including, but not limited to”, unless specifically emphasized otherwise.

Hardware and software environments of a device related to embodiments of the present application will be exemplarily described below.

Exemplary principle description for a signal interaction situation of a diaper-based anomaly analysis and prewarning system provided in an embodiment of the present disclosure is shown below in conjunction with FIG. 1.

Refer to FIG. 1 which is a schematic diagram of signal interaction of a diaper-based anomaly analysis and prewarning system provided in an embodiment of the present disclosure.

In one of embodiments of the present disclosure, the diaper-based anomaly analysis and prewarning system includes a monitoring device 1, a diaper 2 supporting communication connection with the monitoring device 1 through a radio frequency signal, and a portable electronic device 3 supporting communication connection with the monitoring device 1 through a wireless signal (such as a Bluetooth signal, a Wi-Fi signal and/or a wireless communication signal (such as 2G, 3G, 4G and 5G). In the present embodiment, the diaper 2 is configured with an electronic tag component 6, the monitoring device I sends a radio frequency signal, if the diaper 2 is located in a magnetic field induction range of the radio frequency signal, the electronic tag component 6 on the diaper 2 can generate an induction current, and sends a radio frequency signal to the monitoring device 1 based on the generated induction current; the monitoring device 1 receives the radio frequency signal sent by the electronic tag component 6, decodes and analyzes the received radio frequency signal, and determines, according to an analysis result, whether anomaly prewarning information in a preset format needs to be sent to the portable electronic device 3 (example 1, the sent anomaly prewarning information in the preset format can be that “there is urine on a diaper worn by a caregiver, it is recommended to replace the diaper in time”; and example 2, if there is a binding relationship between the monitoring device 1 sending the anomaly prewarning information and specific position information (such as bed 03), the sent anomaly prewarning information in the preset format can be that “there is urine on a diaper worn by a person on bed 03, it is recommended to replace the diaper in time”), and if the anomaly prewarning information needs to be sent, the anomaly prewarning information in the preset format is directly sent to the portable electronic device 3. In the present embodiment, the monitoring device 1 can be paired with the portable electronic device 3 by a Wi-Fi device or a Bluetooth module, and the monitoring device 1 directly sends the anomaly prewarning information to the paired portable electronic device 3.

In another embodiment of the present disclosure, the diaper-based anomaly analysis and prewarning system includes a monitoring device 1, a diaper 2 supporting communication connection with the monitoring device 1 through a radio frequency signal, a background server 5 supporting communication connection with the monitoring device 1 through the Internet (such as a traditional Internet or a mobile Internet), and a portable electronic device 3 supporting communication connection with the background server 5 through a wireless signal. In the present embodiment, the diaper 2 is configured with an electronic tag component 6, the monitoring device 1 sends a radio frequency signal, if the diaper 2 is located in a magnetic field induction range of the radio frequency signal, the electronic tag component 6 on the diaper 2 can generate an induction current, and sends a radio frequency signal to the monitoring device 1 based on the generated induction current; the monitoring device 1 receives the radio frequency signal sent by the electronic tag component 6, decodes and analyzes the received radio frequency signal, and determines, according to an analysis result, whether anomaly prewarning information in a preset format needs to be sent, and if the anomaly prewarning information needs to be sent, a prewarning information sending request is sent to the background server 5; and after receiving the prewarning information sending request, the background server 5 sends the anomaly prewarning information in the preset format to the portable electronic device 3 in response to the prewarning information sending request. In the present embodiment, the monitoring device 1 can be registered and paired with the portable electronic device 3 in the background server 5, and the background server 5 sends the anomaly prewarning information to the portable electronic device 3 registered and paired with the monitoring device 1 in response to the prewarning information sending request.

It should be noted that in an actual usage scenario, the monitoring device 1 can be placed on a specific position of a mattress (with reference to an example of a position where the monitoring device is placed in FIG. 6), or placed on any other applicable positions close to the mattress as long as the normal radio frequency signal communication between the monitoring device 1 and the radio frequency electronic tag on the diaper placed on any position of the mattress is ensured, which will be no longer repeated herein.

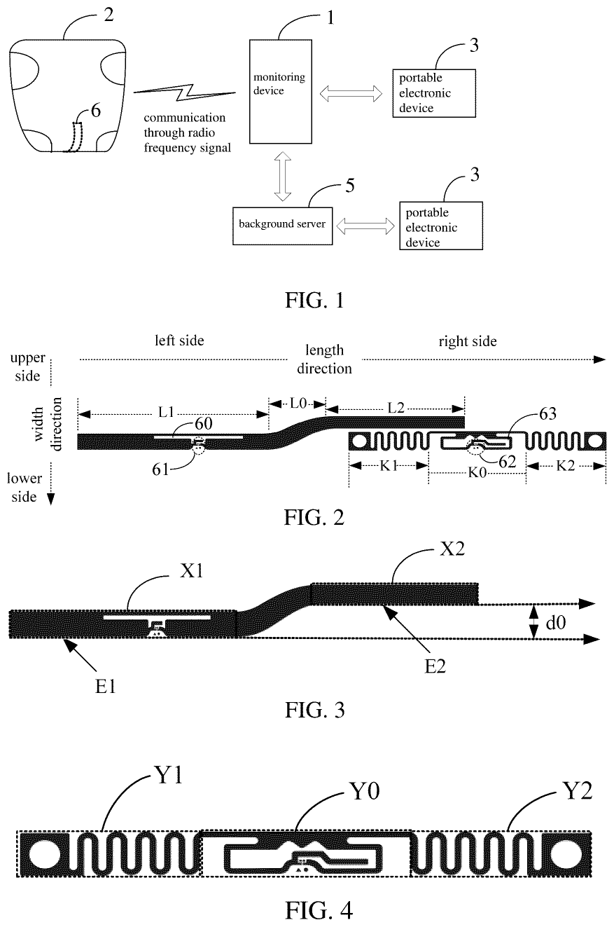

A structure of an electronic tag component 6 mounted on a diaper 2 will be exemplarily described below in conjunction with FIGS. 2 to 4.

Refer to FIG. 2 which is a schematic structural diagram of an electronic tag component 6 which can be mounted on a diaper 2 shown in FIG. 1 in an embodiment of the present disclosure.

The electronic tag component 6 includes a first radio frequency electronic tag for detecting a first radio frequency signal representing that the diaper 2 is located in a perceptible area and a second radio frequency electronic tag for detecting a second radio frequency signal representing the existence of a liquid (such as urine), the first radio frequency electronic tag is a liquid-insensitive electronic tag, and the second radio frequency electronic tag is a liquid-sensitive electronic tag.

It should be noted that the liquid-sensitive electronic tag refers to an electronic tag sending a radio frequency signal of which an intensity value is linearly attenuated relative to a liquid humidity value in a humid working environment, the greater the humidity value is, the smaller the intensity value of the sent radio frequency signal is, and when the humidity value of the working environment tends to 100%, the intensity value of the sent radio frequency signal tends to 0; and the liquid-insensitive electronic tag refers to an electronic tag sending a radio frequency signal of which an intensity value is not attenuated or weakly attenuated relative to a liquid humidity value in a humid working environment, for example, for an ultrahigh-frequency (such as 860-960 MHZ) radio frequency electronic tag based on a slot antenna, when the humidity value of the working environment changes within an interval [0%, 100%], a variation amplitude of the intensity value of the sent radio frequency signal is smaller than a preset value (such as 5 dB).

In the present embodiment, the first radio frequency electronic tag is a radio frequency electronic tag based on a slot antenna (refer to FIG. 3), and the second radio frequency electronic tag is a radio frequency electronic tag based on an electric small loop antenna and half-wave dipole antennae (refer to FIG. 4). The first radio frequency electronic tag includes a first conductive substrate, and the first conductive substrate includes a first substrate segment L1, a second substrate segment L2, and a transition substrate segment L0 electrically connected to the first substrate segment L1 and the second substrate segment L2; the first substrate segment L1 is provided with a linear slot 60 and a first tag chip 61 disposed on one side of the slot 60; the first substrate segment L1 and the second substrate segment L2 are horizontally misaligned in a length direction, and the second substrate segment L2 is located on an upper side of the first substrate segment L1 in a width direction (the first substrate segment L1, the transition substrate segment L0, the second substrate segment L2, the slot 60 and the first tag chip 61 form a radio frequency signal slot antenna with a special shape). The second radio frequency electronic tag includes a first half-wave dipole antenna K1, a second half-wave dipole antenna K2, and a transition antenna segment K0 electrically connected to the first half-wave dipole antenna K1 and the second half-wave dipole antenna K2, the transition antenna segment K0 is provided with an electric small loop antenna 63, the electric small loop antenna 63 is provided with a second tag chip 62, and the first half-wave dipole antenna K1, the second half-wave dipole antenna K2 and the transition antenna segment K0 are located in the same straight line in the length direction; and the second substrate segment L2 is located on an upper side of the first half-wave dipole antenna K1, the second substrate segment L2 and the first half-wave dipole antenna K1 are parallel in the length direction, and the first substrate segment L1 and the first half-wave dipole antenna K1 are located in the same straight line in the length direction. The above-mentioned shape and structure have the advantages that: the difficulty in batch manufacture is lowered, the manufacturing efficiency is increased, the physical resistance of the electronic tag component 6 to wrinkles is effectively improved, it is ensured that the single tag has a sufficient antenna area range to ensure antenna efficiency, at the same time, a range of a longitudinal area, occupied by the electronic tag component 6, of the diaper 2, is reduced, centralized detection of signals on key parts (the electronic tag component 6 is usually disposed on a geometric center position, corresponding to a water absorption layer, of the diaper 2) is ensured, shielding influences of a human body on a radio frequency signal in the case that the diaper 2 is used in different postures are reduced, in addition, the usage amount of substrate consumables for the electronic tag can be further reduced.

Preferably, a misalignment distance between the second substrate segment L2 and the first substrate segment L1 which are horizontally misaligned in the length direction is greater than the maximum width of the first half-wave dipole antenna K1 in the width direction (which can reduce mutual interference among signals while ensuring that a sufficient space is vacated at the lower side of the second substrate segment L2 to accommodate the first half-wave dipole antenna K1), and is smaller than two times of the maximum width of the first substrate segment L1 in the width direction, or smaller than two times of the maximum width of the first half-wave dipole antenna in the width direction, or smaller than two times of a larger width value between the maximum width of the first substrate segment L1 in the width direction and the maximum width of the first half-wave dipole antenna in the width direction (which can ensure a moderate spacing between the second substrate segment L2 and the first half-wave dipole antenna K1 in the width direction, further improve the physical resistance of the tag component to the wrinkles, further lower the difficulty in batch manufacture, and further reduce the usage amount of substrate consumables for the electronic tag).

In the present embodiment, the above-mentioned shape and structure of the electronic tag component 6 bring the technical effects: it can be ensured that the second substrate segment L2 and the first half-wave dipole antenna K1 are parallel in the length direction, it can be ensured that a sufficient space is vacated at the lower side of the second substrate segment L2 to accommodate the first half-wave dipole antenna K1, it can be ensured that the first substrate segment L1 and the first half-wave dipole antenna K1 are located in the same straight line in the length direction, and signal interference between the first radio frequency electronic tag and the second radio frequency electronic tag is effectively reduced; and resistance to signal interference can be achieved, at the same time, a moderate spacing between the second substrate segment L2 and the first half-wave dipole antenna K1 in the width direction can be ensured, the difficulty in batch manufacture can be lowered, the manufacturing efficiency can be increased, length and width spaces of the diaper 2 occupied by the electronic tag component 6 can be effectively reduced, the physical resistance of the electronic tag component 6 to the wrinkles can be improved, and an effective balance is formed between the signal interference resistance and the physical resistance to the wrinkles, so that the electronic tag component 6 is applicable to an application scenario of liquid anomaly analysis and prewarning for the diaper 2 based on various radio frequency signals.

The statement “the first substrate segment L1 and the second substrate segment L2 are horizontally misaligned in a length direction” is further explained as that: a minimum rectangular surrounding box X1 (such as a rectangular box X1 shown by dotted lines in FIG. 3) is made for a surface of the first substrate segment L1, a minimum rectangular surrounding box X2 (such as a rectangular box X2 shown by dotted lines in FIG. 3) is made for a surface of the second substrate segment L2, and a center line of the minimum rectangular surrounding box X1 in the length direction and a center line of the minimum rectangular surrounding box X2 in the length direction are parallel, but are not located in the same straight line, in this case, it is referred to as “the first substrate segment L1 and the second substrate segment L2 are horizontally misaligned in a length direction”.

The statement “a misalignment distance between the second substrate segment L2 and the first substrate segment L1 which are horizontally misaligned in the length direction” is further explained as a vertical distance between a lower box edge (such as a lower box edge E1 of the rectangular box X1 shown by dotted lines in FIG. 3) of the minimum rectangular surrounding box X1 and a lower box edge (such as a lower box edge E2 of the rectangular box X2 shown by dotted lines in FIG. 3) of the minimum rectangular surrounding box X2 in the width direction (for example, a vertical distance do between an extension line of the lower box edge E1 in the length direction and an extension line of the lower box edge E2 in the length direction in FIG. 3 in the width direction is the vertical distance).

The statement “the first half-wave dipole antenna K1, the second half-wave dipole antenna K2 and the transition antenna segment K0 are located in the same straight line in the length direction” is further explained as that: a minimum rectangular surrounding box Y1 (such as a rectangular box Y1 shown by dotted lines in FIG. 4) is made for a surface of the first half-wave dipole antenna K1, a minimum rectangular surrounding box Y2 (such as a rectangular box Y2 shown by dotted lines in FIG. 4) is made for a surface of the second half-wave dipole antenna K2, a minimum rectangular surrounding box Y0 (such as a rectangular box Y0 shown by dotted lines in FIG. 4) is made for a surface of the transition antenna segment K0, and a center line of the minimum rectangular surrounding box Y0 in the length direction, a center line of the minimum rectangular surrounding box Y1 in the length direction and a center line of the minimum rectangular surrounding box Y2 in the length direction are located in the same straight line, in this case, it is referred to as “the first half-wave dipole antenna K1, the second half-wave dipole antenna K2 and the transition antenna segment K0 are located in the same straight line in the length direction”.

The statement “the second substrate segment L2 and the first half-wave dipole antenna K1 are parallel in the length direction, and the first substrate segment L1 and the first half-wave dipole antenna K1 are located in the same straight line in the length direction” is further explained as that: the center line of the minimum rectangular surrounding box X2 in the length direction and the center line of the minimum rectangular surrounding box Y1 in the length direction are parallel, and the center line of the minimum rectangular surrounding box X1 in the length direction and the center line of the minimum rectangular surrounding box Y1 in the length direction are located in the same straight line, in this case, it is referred to as “the second substrate segment L2 and the first half-wave dipole antenna K1 are parallel in the length direction, and the first substrate segment L1 and the first half-wave dipole antenna K1 are located in the same straight line in the length direction”.

The statement “the maximum width of the first half-wave dipole antenna K1 or the first substrate segment L1 in the width direction” is further explained as that: a minimum rectangular surrounding box (such as the rectangular box Y1 shown by dotted lines in FIG. 4 or the rectangular box X1 shown by dotted lines in FIG. 3) is made for a surface of the first half-wave dipole antenna K1 or the first substrate segment L1, and the width of the minimum rectangular surrounding box is “the maximum width in the width direction” of a corresponding part.

The statement “a larger width value between the maximum width of the first substrate segment L1 in the width direction and the maximum width of the first half-wave dipole antenna in the width direction” is further explained as that: if the maximum width of the first substrate segment L1 in the width direction is greater than or equal to the maximum width of the first half-wave dipole antenna in the width direction, the maximum width of the first substrate segment L1 in the width direction is the larger width value, or else, the maximum width of the first half-wave dipole antenna in the width direction is the larger width value.

It should be noted that any technical features, such as “parallel”, “vertical”, and “located in the same straight line”, in any mathematical sense related to all the embodiments of the present disclosure are only for simplifying description, but do not mean that actual shapes and structures of corresponding parts of a product should be absolutely the same as the technical features described in the mathematical sense during the actual manufacture and use of the product. It should be known by the skilled in the art that, due to the restriction of various physical conditions, natural conditions, technical conditions, etc., there must be reasonable errors between the actual shapes and structures of the corresponding parts of the product and ideal literal features during actual industrial application, the existence of these reasonable errors is an inevitable result conforming to the natural law, and it is necessary to take these reasonable errors into account when the features in the technical solution disclosed by the present disclosure are interpreted.

It should be noted that a manufacturing process of the electronic tag component 6 is the same as an existing manufacturing process, and it is unnecessary to adopt a special manufacturing process. For example, adoptable manufacturing processes include: chip packaging is respectively performed on tag chips of the first radio frequency electronic tag and the second radio frequency electronic tag (for example, a chip can be packaged in a plastic module by using a welding technology to prevent the chip from being affected by an external environment, and related manufacturing processes are existing processes, which will be no longer repeated herein); tag antennae of the first radio frequency electronic tag and the second radio frequency electronic tag are respectively manufactured (for example, it can be completed by printing, that is, a conductive material is printed on a plastic substrate and is clipped and/or bent to form an antenna with a specific shape, and related manufacturing processes are existing processes, which will be no longer repeated herein); packaged chips of the first radio frequency electronic tag and the second radio frequency electronic tag are fitted to the manufactured tag antennae to form the complete first radio frequency electronic tag and the second radio frequency electronic tag (for example, the packaged chips can be fixed together with the tag antennae by using a binder to respectively form an integrated single tag, and related manufacturing processes are existing processes, which will be no longer repeated herein); based on a design structure of the electronic tag component 6, the complete first radio frequency electronic tag and second radio frequency electronic tag are respectively fitted to a common substrate in order to form the complete electronic tag component 6 (for example, the complete first radio frequency electronic tag and second radio frequency electronic tag can be respectively fitted to the common substrate by using the binder to form an integrated tag, during batch manufacture, the first radio frequency electronic tag and the second radio frequency electronic tag can be used as parts to be rapidly mounted on the complete common substrate, a tearable structure of a single electronic tag component 6 can be processed on the complete common substrate while batch mounting or after mounting, and these processes are all existing processes, which will be no longer repeated herein); and subsequent processes (such as testing, coding, and packaging, and related processes are all existing processes, which will be no longer repeated herein) are performed.

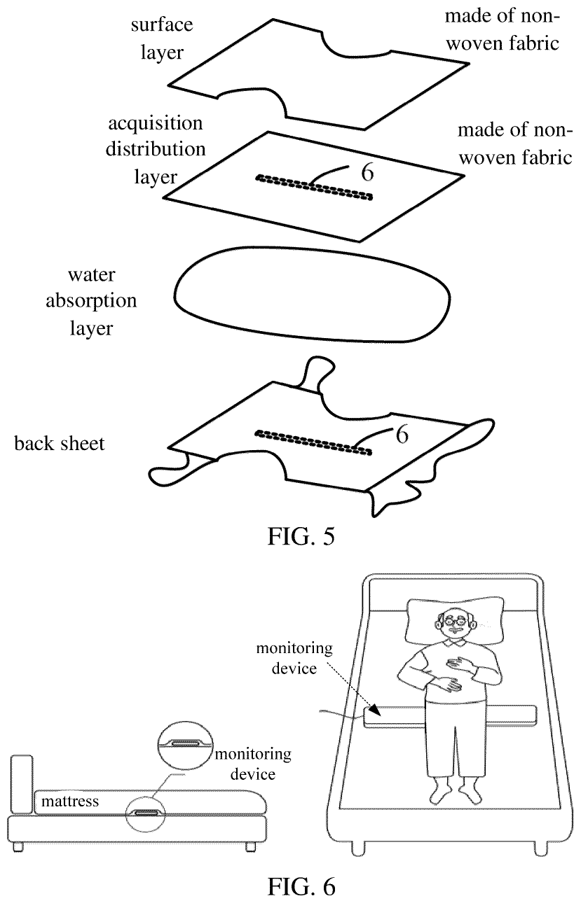

A structure of a diaper 2 will be exemplarily described below in conjunction with FIG. 5.

Refer to FIG. 5 which is a schematic diagram of a layered structure of the diaper 2 shown in FIG. 1. In one of embodiments of the present disclosure, the diaper 2 includes a surface layer, an acquisition distribution layer, a water absorption layer, and a back sheet, the electronic tag component 6 can be fixedly mounted on the acquisition distribution layer and/or the back sheet (a mounting position on the acquisition distribution layer and/or the back sheet can be any applicable position, such as a position close to a side and a position near a geometric center, and in order to improve the sensitivity of urine anomaly analysis, it is preferably mounted on the position near the geometric center).

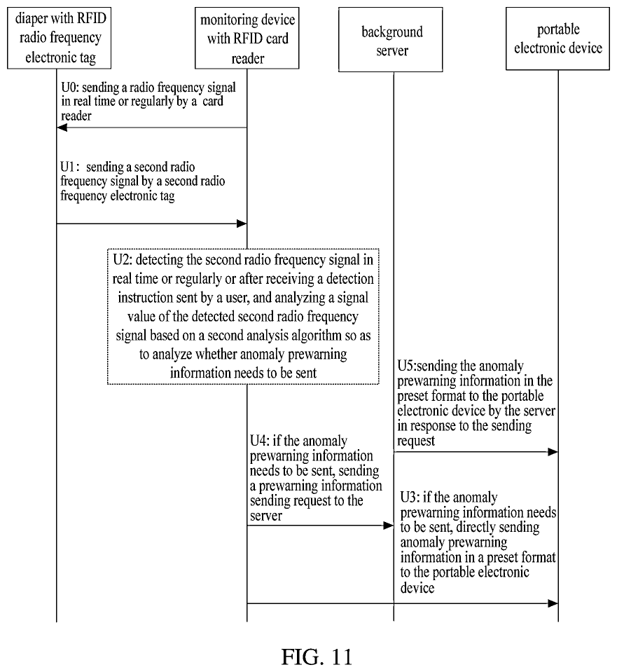

It should be noted that the layered structure of the diaper 2 shown in FIG. 5 is only one of embodiments of the present disclosure; in other embodiments of the present disclosure, the diaper 2 can further include any other applicable material layers; when the layered structure of the diaper 2 changes, the mounting position of the electronic tag component 6 can also be adaptively adjusted as long as it is ensured that the electronic tag component 6 is mounted on a material layer which can be in rapid contact with a humid environment. In addition, in other embodiments of the present disclosure, in order to reduce the use cost of the diaper, the electronic tag component 6 can be replaced with a single liquid-sensitive electronic tag (such as the second radio frequency electronic tag), and an application method of the replaced single liquid-sensitive electronic tag can refer to the following exemplary description for FIGS. 11 to 13, which will be no longer repeated herein.

It should be noted that a manufacturing process of the diaper 2 is the same as an existing manufacturing process, and it is unnecessary to adopt a special manufacturing process. For example, adoptable manufacturing processes include: an absorption core is manufactured (for example, a non-woven fabrics, a hot melt adhesive and water absorbent resin can be superimposed together in a certain proportion, and then hot-pressed at high temperature through a hot press to form an absorption core, related processes are all existing processes, which will be no longer repeated herein); the surface layer, the acquisition distribution layer and the back sheet are processed (related processes are all existing processes, and the electronic tag component 6 can be mounted on the acquisition distribution layer and/or the back sheet by binding by using an ordinary adhesive, or binding by using a magic sticker, or sewing an edge of a substrate of the electronic tag component 6 to the acquisition distribution layer and/or the back sheet, which will be no longer repeated herein); and assembling and packaging are performed (related processes are all existing processes, for example, an assembling process can be that the absorption core, the acquisition distribution layer, the surface layer and the back sheet are superimposed together in a determined order, and then cut, sealed, drilled or processed in other ways through a large production line to form a complete diaper).

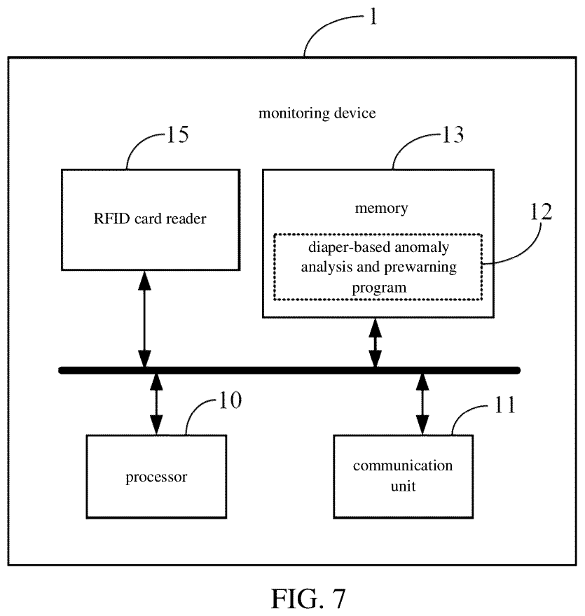

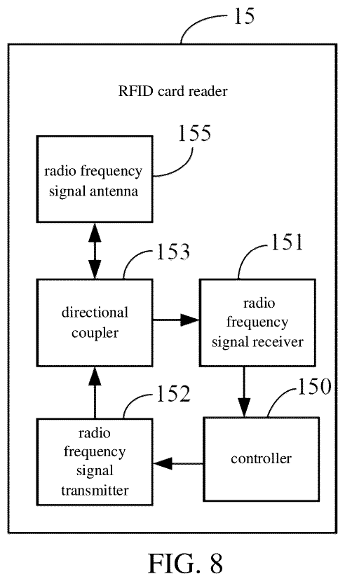

A structure of a monitoring device 1 matched with the diaper 2 will be exemplarily described below in conjunction with FIGS. 7 to 8.

It should be noted that FIGS. 7 to 8 only exemplarily show component structures of a monitoring device land an RFID (Radio Frequency Identification) card reader 15 in one of embodiments of the present disclosure. It can be understood by the skilled in the art that the component structures shown in FIGS. 7 to 8 do not constitute structural limitations on the monitoring device 1 and the RFID card reader 15. In other embodiments of the present disclosure, the monitoring device 1 and/or the RFID card reader 15 can include more or fewer components than those shown in the figures, or include more or fewer specific component combinations than those shown in the figures, or arrangement structures of the components of the monitoring device 1 and/or the RFID card reader 15 can be different from those shown in the figures, which will be no longer repeated herein.

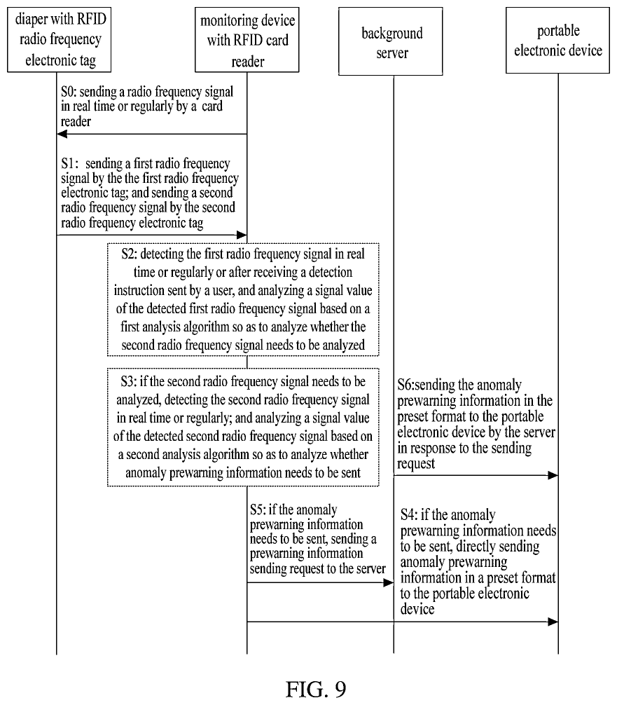

Refer to FIG. 7 which is a schematic diagram of an internal structure of the monitoring device 1 shown in FIG. 1. In one of embodiments of the present disclosure, the monitoring device 1 includes a communication unit 11, a memory 13, an RFID card reader 15, and a processor 10 in communication connection to the communication unit 11, the memory 13 and the RFID card reader 15, respectively. The memory 13 stores a diaper-based anomaly analysis and prewarning program 12 callable and executable for the processor 10, and when the diaper-based anomaly analysis and prewarning program 12 is called and executed by the processor 10, steps in which the monitoring device 1 participates in a flow diagram shown in any figure file in FIGS. 9 to 13 are performed, and the steps refer to the following description for FIGS. 9 to 13, which will be no longer repeated herein.

The memory 13 at least includes one type of computer-readable storage medium including a flash memory, a hard disk, a multimedia card, a card-type memory (such as an SD or a DX memory), a random access memory (RAM), a static random access memory (SRAM), a read-only memory (ROM), an electrically erasable programmable read-only memory (EEPROM), a programmable read-only memory (PROM), a magnetic memory, a magnetic disk, an optical disk, etc. In some embodiments, the memory 13 can be an internal storage unit of the monitoring device 1, such as a hard disk or an internal memory of the monitoring device 1. In other embodiments, the memory 13 can also be an outsourcing storage device of the monitoring device 1, such as a plug-in hard disk, a Smart Media Card (SMC), an Secure Digital (SD) card and a Flash Card which are equipped on the monitoring device 1. Of course, the memory 13 can further include both the internal storage unit and the outsourcing storage device of the monitoring device 1. In the present embodiment, the memory 13 is usually used for storing an operating system and various applications installed in the monitoring device 1, such as the diaper-based anomaly analysis and prewarning program 12. In addition, the memory 13 can be further used for temporarily storing various data which has been outputted or is to be outputted.

In some embodiments, the processor 10 can be a Central Processing Unit (CPU), a controller, a micro-controller, a microprocessor, or other data processing chips. The processor 10 is usually used for controlling the overall operation of the monitoring device 1. In the present embodiment, the processor 10 is used for operating a program code or processed data stored in the memory 13, such as the diaper-based anomaly analysis and prewarning program 12 stored in the memory 13.

In some embodiments, the communication unit 11 can include a mobile communication module and/or a wireless communication module, and the mobile communication module can provide wireless communication solutions applied to the monitoring device 1 and including 2G/3G/4G/5G, etc. The mobile communication module can include at least one filter, a switch, a power amplifier, a Low Noise Amplifier (LNA), etc. The mobile communication module can receive electromagnetic waves through antennae (unshown in the figure) and process, such as filter and amplify, the received electromagnetic waves which are transmitted to a modulation and demodulation processor for demodulation. Signals modulated by the modulation and demodulation processor can be further amplified by the mobile communication module, and can be converted into electromagnetic waves by antennae so as to be radiated. The wireless communication module can provide wireless communication solutions applied to the monitoring device 1 and including Wireless Local Area Networks (WLAN) (such as a wireless fidelity (Wi-Fi) network), bluetooth (BT), and Near Field Communication (NFC). The wireless communication module can be one or more devices integrated with at least one communication processing module. The wireless communication module receives electromagnetic waves through antennae (unshown in the figure), performs signal frequency modulation and filtration on the electromagnetic waves, and sends the processed signals to the processor 10. The wireless communication module can further receive to-be-sent signals from the processor 10, perform frequency modulation and amplification on the signals, and convert, through antennae, the signals into electromagnetic waves to be radiated.

Refer to FIG. 8 which is a schematic diagram of an internal structure of an RFID card reader 15 shown in FIG. 5. In one of embodiments of the present disclosure, the RFID card reader 15 includes a radio frequency signal antenna 155, a directional coupler 153, a radio frequency signal receiver 151 and a radio frequency signal transmitter 152 connected to the directional coupler 153 by signals, and a controller 150 connected to the radio frequency signal receiver 151 and the radio frequency signal transmitter 152 by signals. The radio frequency signal antenna 155 can adopt any existing applicable type of radio frequency signal antenna (such as an antenna which is the same as an antenna in a first radio frequency electronic tag and/or an antenna in a second radio frequency electronic tag in type and is matched with the same in working parameters, such as an antenna disclosed in a Chinese patent application No. “CN202321933117.6”, entitled “Ultrahigh-Frequency Composite RFID Slot Antenna”), which will be no longer repeated herein. The radio frequency signal receiver 151 and the radio frequency signal transmitter 152 can adopt any existing applicable type of functional device (for example, the radio frequency signal receiver 151 can include a demodulator, a filter, an intermediate-frequency amplifier, an analog-to-digital converter (ADC), etc.; the radio frequency signal transmitter 152 can include a radio frequency oscillator, a modulator, a power amplifier (PA), etc.; and an output end of the radio frequency signal transmitter 152 and an input end of the radio frequency signal receiver 151 are connected to the radio frequency signal antenna 155 through the directional coupler 153), which will be no longer repeated herein.

In one of embodiments of the present disclosure, the controller 150 is a separate microprocessing chip. In other embodiments of the present disclosure, the controller 150 of the RFID card reader 15 is not a separate chip, can be understood as a part of the processor 10, and is integrated with the processor 10 to become a separate processor, which will be no longer repeated herein.

A flow of a diaper-based anomaly analysis and prewarning method will be exemplarily described below in conjunction with cooperation among a monitoring device 1, a diaper 2, a portable electronic device 3 and/or a background server 5 in FIGS. 9 to 13.

Refer to FIG. 9 which is a schematic flow diagram of a diaper-based anomaly analysis and prewarning method provided in a first embodiment of the present disclosure. In the present embodiment, the diaper-based anomaly analysis and prewarning method includes the following steps:

SO, the monitoring device 1 sends a radio frequency signal in real time or regularly based on the RFID card reader 15 (for example, the controller 150 controls the radio frequency signal transmitter 150 to send the radio frequency signal through the directional coupler 153 and the radio frequency signal antenna 155);
S1, a radio frequency signal slot antenna in the first radio frequency electronic tag of the electronic tag component 6 sends a first radio frequency signal when generating electromagnetic induction with the radio frequency signal, and an electric small loop antenna and half-wave dipole antennae in the second radio frequency electronic tag send a second radio frequency signal when generating electromagnetic induction with the radio frequency signal;
S2, the monitoring device 1 detects the first radio frequency signal based on the RFID card reader 15 in real time or regularly or after receiving a detection instruction sent by a user (for example, the detection instruction can be sent through an excitation button configured on an electronic device applicable to the method, “such as a physical button configured on the electronic device and named ‘start to detect’”, or sent through a touch control button displayed on a display configured on an electronic device applicable to the method and named ‘start to detect’, or sent to an electronic device applicable to the method through other electronic devices “such as a mobile phone and a tablet personal computer”, which will be no longer repeated herein), and analyzes a signal value of the detected first radio frequency signal based on a first analysis algorithm so as to analyze whether the second radio frequency signal needs to be analyzed;
S3, if the second radio frequency signal needs to be analyzed, the second radio frequency signal is detected in real time or regularly based on the RFID card reader 15, and a signal value of the detected second radio frequency signal is analyzed based on a second analysis algorithm so as to analyze whether anomaly prewarning information needs to be sent; and
S4, if the anomaly prewarning information needs to be sent, the monitoring device 1 directly sends anomaly prewarning information in a preset format to the portable electronic device 3.

In one of other embodiments of the present disclosure, after step S3, the diaper-based anomaly analysis and prewarning method includes the following steps:
S5, if the anomaly prewarning information needs to be sent, the monitoring device 1 sends a prewarning information sending request to the background server 5; and
S6, the background server 5 sends the anomaly prewarning information in the preset format to the portable electronic device 3 in response to the sending request.

In one of embodiments of the present disclosure, the first analysis algorithm includes:
a signal value of a first radio frequency signal detected currently is analyzed so as to analyze whether the signal value of the first radio frequency signal is greater than a first preset value (such as 35 dB);
if the signal value of the first radio frequency signal detected currently is greater than the first preset value, it is determined that the second radio frequency signal needs to be analyzed; and
if the signal value of the first radio frequency signal detected currently is smaller than or equal to the first preset value, it is determined that the second radio frequency signal does not need to be analyzed.

In order to prevent misjudgment, improve the accuracy of excitation conditions for detecting the second radio frequency signal, and reduce energy consumption and computing resources, preferably, in another embodiment of the present disclosure, the first analysis algorithm includes:
a signal value of a first radio frequency signal detected currently is analyzed so as to analyze whether the signal value of the first radio frequency signal is greater than a first preset value (such as 35 dB);
if the signal value of the first radio frequency signal detected currently is greater than the first preset value, signal values of a first preset number (such as two or three) of first radio frequency signals detected continuously (for example, the first preset number of first radio frequency signals detected continuously include the first radio frequency signal detected currently and K first radio frequency signals previously detected continuously, wherein K is a positive integer, and the first preset number is equal to K+1) are analyzed so as to analyze whether a variation amplitude value (i.e., a variation amplitude value obtained by subtracting the minimum signal value by the maximum signal value in the signal values of the first preset number of first radio frequency signals detected continuously) of the signal values of the first preset number of first radio frequency signals detected continuously is greater than a second preset value (such as 5 dB);
if the variation amplitude value is smaller than or equal to the second preset value, it is determined that the second radio frequency signal needs to be analyzed; and
if the variation amplitude value is greater than the second preset value, it is determined that the second radio frequency signal does not need to be analyzed.

In one of embodiments of the present disclosure, the second analysis algorithm includes:
a signal value of a second radio frequency signal detected currently is analyzed so as to analyze whether the signal value of the second radio frequency signal detected currently is greater than a third preset value (such as 40 dB, the third preset value can be the same as or different from the first preset value, and preset values limited in all the embodiments of the present disclosure are all exemplary operating parameters in technical solutions disclosed by the present disclosure, and are not intended to limit that the technical solutions disclosed by the present disclosure necessarily adopt exemplary values);
if the signal value of the second radio frequency signal detected currently is greater than the third preset value, it is determined that the anomaly prewarning information does not need to be sent; and
if the signal value of the second radio frequency signal detected currently is smaller than or equal to the third preset value, it is determined that the anomaly prewarning information needs to be sent.

In order to prevent misjudgment, improve the accuracy of excitation conditions for sending the anomaly prewarning information, and reduce computing resources, preferably, in another embodiment of the present disclosure, the second analysis algorithm includes:
a signal value of a second radio frequency signal detected currently is analyzed so as to analyze whether the signal value of the second radio frequency signal detected currently is greater than a third preset value;
if the signal value of the second radio frequency signal detected currently is smaller than or equal to the third preset value, signal values of a second preset number (such as two or three) of second radio frequency signals detected continuously (for example, the second preset number of second radio frequency signals detected continuously include the second radio frequency signal detected currently and M first radio frequency signals previously detected continuously, wherein M is a positive integer, the second preset number is equal to M+1, and M and K can be the same or different) are analyzed so as to analyze whether there are second radio frequency signals of which signal values are greater than the third preset value in the second preset number of second radio frequency signals detected continuously;
if there are the second radio frequency signals of which the signal values are greater than the third preset value, the step is returned to perform the step that the second radio frequency signal is detected in real time or regularly and steps after the step; and
if the signal values of all the second radio frequency signals are smaller than or equal to the third preset value, it is determined that the anomaly prewarning information needs to be sent.

In order to prevent anomaly prewarning information sending errors caused by the interference of a current position of an electronic tag to signal intensity and the interference of different postures of a human body to signal intensity, improve the accuracy of real urine detection, and then improve the accuracy of excitation conditions for sending the anomaly prewarning information, preferably, in other embodiments of the present disclosure, the second analysis algorithm includes:
a signal value of a second radio frequency signal detected currently is analyzed so as to analyze whether the signal value of the second radio frequency signal detected currently is greater than a third preset value;
if the signal value of the second radio frequency signal detected currently is smaller than or equal to the third preset value, signal values of a second preset number of second radio frequency signals detected continuously are analyzed so as to analyze whether there are second radio frequency signals of which signal values are greater than the third preset value in the second preset number of second radio frequency signals detected continuously;
if there are the second radio frequency signals of which the signal values are greater than the third preset value, the step is returned to perform the step that the second radio frequency signal is detected in real time or regularly and steps after the step;
if the signal values of all the second radio frequency signals are smaller than or equal to the third preset value, the signal values of the second preset number of second radio frequency signals detected continuously are analyzed so as to analyze whether a variation amplitude value of the signal values of the second preset number of second radio frequency signals detected continuously is greater than a fourth preset value (such as 25 dB);
if the variation amplitude value is smaller than or equal to the fourth preset value, the step is returned to perform the step that the second radio frequency signal is detected in real time or regularly and steps after the step; and
if the variation amplitude value is greater than the fourth preset value, it is determined that the anomaly prewarning information needs to be sent.

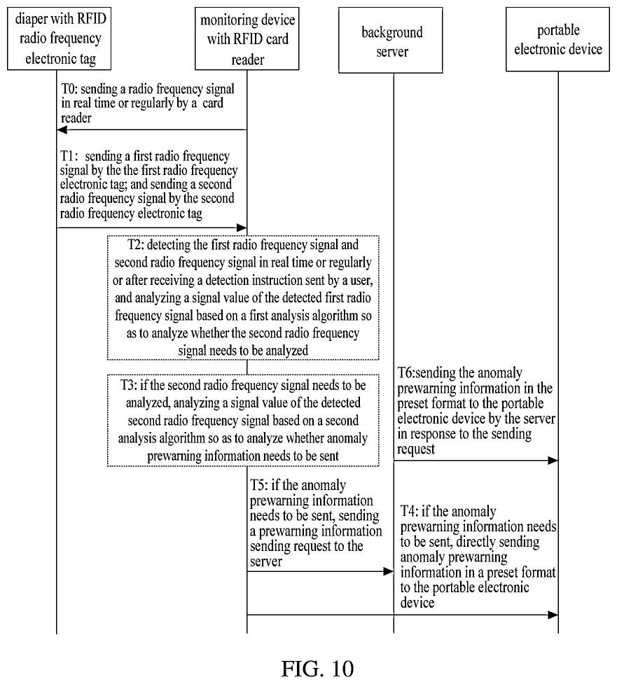

Refer to FIG. 10 which is a schematic flow diagram of a diaper-based anomaly analysis and prewarning method provided in a second embodiment of the present disclosure. In the present embodiment, the diaper-based anomaly analysis and prewarning method includes the following steps:
T0, the monitoring device 1 sends a radio frequency signal in real time or regularly based on the RFID card reader 15;
T1, a radio frequency signal slot antenna in the first radio frequency electronic tag of the electronic tag component 6 sends a first radio frequency signal when generating electromagnetic induction with the radio frequency signal, and an electric small loop antenna and half-wave dipole antennae in the second radio frequency electronic tag send a second radio frequency signal when generating electromagnetic induction with the radio frequency signal;
T2, the monitoring device 1 detects the first radio frequency signal and the second radio frequency signal based on the RFID card reader 15 in real time or regularly or after receiving a detection instruction sent by a user, and analyzes a signal value of the detected first radio frequency signal based on a first analysis algorithm so as to analyze whether the second radio frequency signal needs to be sent;
T3, if the second radio frequency signal needs to be analyzed, the monitoring device 1 analyzes a signal value of the detected second radio frequency signal based on a second analysis algorithm so as to analyze whether anomaly prewarning information needs to be sent; and
T4, if the anomaly prewarning information needs to be sent, the monitoring device 1 directly sends anomaly prewarning information in a preset format to the portable electronic device 3.

In one of other embodiments of the present disclosure, after step T3, the diaper-based anomaly analysis and prewarning method includes the following steps:
T5, if the anomaly prewarning information needs to be sent, the monitoring device 1 sends a prewarning information sending request to the background server 5; and
T6, the background server 5 sends the anomaly prewarning information in the preset format to the portable electronic device 3 in response to the sending request.

Refer to FIG. 11 which is a schematic flow diagram of a diaper-based anomaly analysis and prewarning method provided in a third embodiment of the present disclosure. In the present embodiment, the electronic tag component 6 in the diaper 2 is replaced with a second radio frequency electronic tag, and the diaper-based anomaly analysis and prewarning method includes the following steps:
U0, the monitoring device 1 sends a radio frequency signal in real time or regularly based on the RFID card reader 15;
U1, an electric small loop antenna and half-wave dipole antennae in the second radio frequency electronic tag send a second radio frequency signal when generating electromagnetic induction with the radio frequency signal;
U2, the monitoring device 1 detects a second radio frequency signal based on the RFID card reader 15 in real time or regularly or after receiving a detection instruction sent by a user, and analyzes a signal value of the detected second radio frequency signal based on a second analysis algorithm so as to analyze whether anomaly prewarning information needs to be sent; and
U3, if the anomaly prewarning information needs to be sent, the monitoring device 1 directly sends anomaly prewarning information in a preset format to the portable electronic device 3.

In one of other embodiments of the present disclosure, after step U2, the diaper-based anomaly analysis and prewarning method includes the following steps:
U4, if the anomaly prewarning information needs to be sent, the monitoring device 1 sends a prewarning information sending request to the background server 5; and
U5, the background server 5 sends the anomaly prewarning information in the preset format to the portable electronic device 3 in response to the sending request.

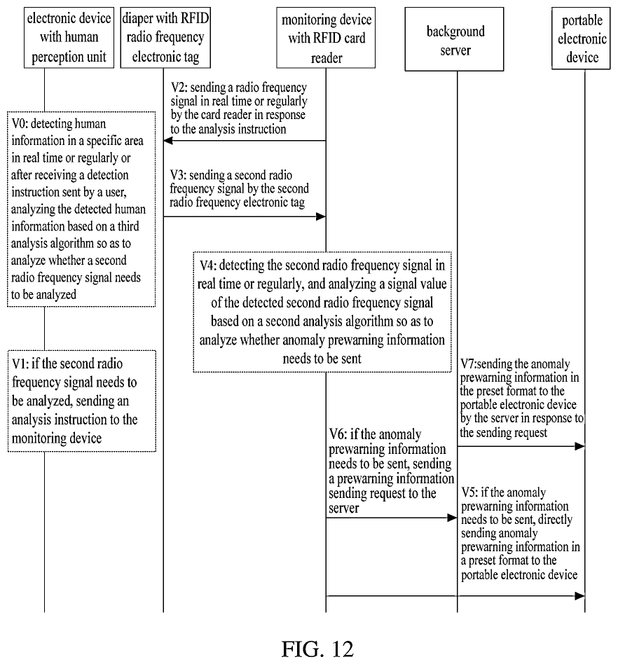

Refer to FIG. 12 which is a schematic flow diagram of a diaper-based anomaly analysis and prewarning method provided in a fourth embodiment of the present disclosure. In an embodiment of the present disclosure, the electronic tag component 6 in the diaper 2 is replaced with a second radio frequency electronic tag, the diaper-based anomaly analysis and prewarning system shown in FIG. 1 further includes an electronic device in communication connection to the monitoring device 1 and provided with a human perception unit (for example, the electronic device with the human perception unit can be an existing camera with a human image recognition function, a lens of the camera can be set to point to a baby bed, a central point of a lens picture can be a geometric central point of the baby bed, the lens picture includes a complete picture of the baby bed, and a proportion of the complete picture of the baby bed in the overall lens picture can be greater than 60%), and the diaper-based anomaly analysis and prewarning method includes the following steps:
V0, the electronic device with the human perception unit detects human information in a specific area based on the human perception unit in real time or regularly or after receiving a detection instruction sent by a user, analyzes the detected human information based on a third analysis algorithm so as to analyze whether the second radio frequency signal needs to be analyzed;
V1, if the second radio frequency signal needs to be analyzed, the electronic device with the human perception unit sends an analysis instruction to the monitoring device 1;
V2, the monitoring device 1 sends a radio frequency signal in real time or regularly based on the RFID card reader 15 in response to the analysis instruction;
V3, an electric small loop antenna and half-wave dipole antennae in the second radio frequency electronic tag send a second radio frequency signal when generating electromagnetic induction with the radio frequency signal;
V4, the monitoring device 1 detects the second radio frequency signal in real time or regularly based on the RFID card reader 15, and analyzes a signal value of the detected second radio frequency signal based on a second analysis algorithm so as to analyze whether anomaly prewarning information needs to be sent; and
V5, if the anomaly prewarning information needs to be sent, the monitoring device 1 directly sends anomaly prewarning information in a preset format to the portable electronic device 3.

In one of other embodiments of the present discloses, after step V4, the diaper-based anomaly analysis and prewarning method includes the following steps:
V6, if the anomaly prewarning information needs to be sent, the monitoring device 1 sends a prewarning information sending request to the background server 5; and
V7, the background server 5 sends the anomaly prewarning information in the preset format to the portable electronic device 3 in response to the sending request.

In one of other embodiments of the present disclosure, the electronic tag component 6 in the diaper 2 is replaced with a second radio frequency electronic tag, the electronic device with the human perception unit shown in FIG. 12 and the monitoring device 1 shown in FIG. 7 can be the same electronic device, at the moment, the monitoring device 1 further includes the human perception unit in communication connection to the processor 10 (for example, the human perception unit can be an infrared sensor which can be placed on the geometric central position of the baby bed, and the specific area can be a spatial area with the geometric central position of the baby bed as a center of a circle and a radius of 2.5 m), and the diaper-based anomaly analysis and prewarning method includes the following steps:
the monitoring device 1 detects human information in a specific area based on the human perception unit in real time or regularly or after receiving a detection instruction sent by a user, analyzes the detected human information based on a third analysis algorithm so as to analyze whether the second radio frequency signal needs to be analyzed;
if the second radio frequency signal needs to be analyzed, the monitoring device 1 sends a radio frequency signal in real time or regularly based on the RFID card reader 15;
an electric small loop antenna and half-wave dipole antennae in the second radio frequency electronic tag send the second radio frequency signal when generating electromagnetic induction with the radio frequency signal;
the monitoring device 1 detects the second radio frequency signal in real time or regularly based on the RFID card reader 15, and analyzes a signal value of the detected second radio frequency signal based on a second analysis algorithm so as to analyze whether anomaly prewarning information needs to be sent; and
if the anomaly prewarning information needs to be sent, the monitoring device 1 directly sends anomaly prewarning information in a preset format to the portable electronic device 3, or if the anomaly prewarning information needs to be sent, the monitoring device 1 sends a prewarning information sending request to the background server 5, and the background server 5 sends the anomaly prewarning information in the preset format to the portable electronic device 3 in response to the sending request.

In one of embodiments of the present disclosure, the third analysis algorithm includes:
if no human body exists in the specific area, it is determined that the second radio frequency signal does not need to be analyzed;
if a duration when human bodies exist in the specific area is smaller than a preset time (such as 5 min), it is determined that the second radio frequency signal does not need to be analyzed; and
if the duration when the human bodies exist in the specific area is greater than or equal to the preset time, it is determined that the second radio frequency signal needs to be analyzed.

In order to prevent misjudgment, improve the accuracy of excitation conditions for detecting the second radio frequency signal, and reduce energy consumption and computing resources, preferably, in another embodiment of the present disclosure, the third analysis algorithm includes:
if no human body exists in the specific area, it is determined that the second radio frequency signal does not need to be analyzed;
if a duration when human bodies exist in the specific area is greater than or equal to a preset time, it is analyzed whether positions where the human bodies exist change within the duration when the human bodies exist; and
if the positions where the human bodies exist do not change, it is determined that the second radio frequency signal needs to be analyzed; and
if the positions where the human bodies exist change, the step is returned to perform the step that human information is detected in a specific area in real time or regularly and steps thereafter.

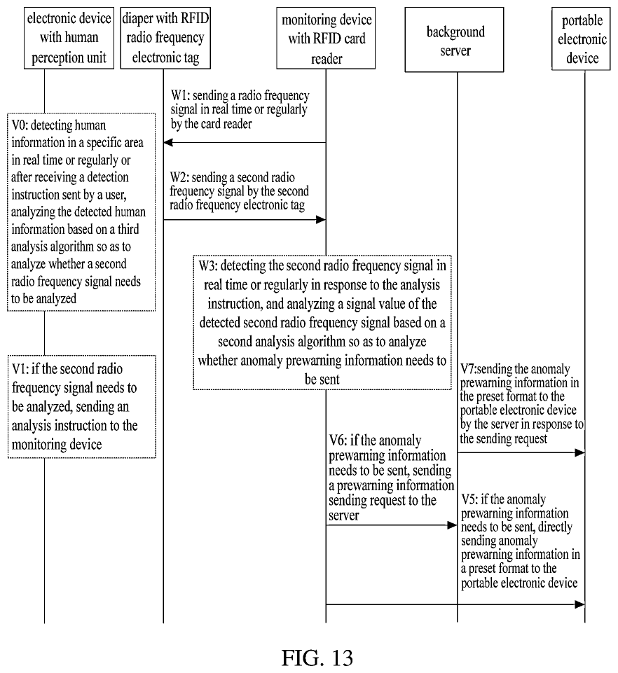

Refer to FIG. 13 which is a schematic flow diagram of a diaper-based anomaly analysis and prewarning method provided in a fifth embodiment of the present disclosure. In an embodiment of the present disclosure, the electronic tag component 6 in the diaper 2 is replaced with a second radio frequency electronic tag, the diaper-based anomaly analysis and prewarning system shown in FIG. 1 further includes an electronic device in communication connection to the monitoring device 1 and provided with a human perception unit, and the diaper-based anomaly analysis and prewarning method includes the following steps:
W1, the monitoring device 1 sends a radio frequency signal in real time or regularly based on the RFID card reader 15;
W2, an electric small loop antenna and half-wave dipole antennae in the second radio frequency electronic tag send a second radio frequency signal when generating electromagnetic induction with the radio frequency signal;
V0, the electronic device with the human perception unit detects human information in a specific area based on the human perception unit in real time or regularly or after receiving a detection instruction sent by a user, analyzes the detected human information based on a third analysis algorithm so as to analyze whether the second radio frequency signal needs to be analyzed;
V1, if the second radio frequency signal needs to be analyzed, the electronic device with the human perception unit sends an analysis instruction to the monitoring device 1;
W3, the second radio frequency signal is detected in real time or regularly in response to the analysis instruction, and analyzes a signal value of the detected second radio frequency signal based on a second analysis algorithm so as to analyze whether anomaly prewarning information needs to be sent; and
V5, if the anomaly prewarning information needs to be sent, the monitoring device 1 directly sends anomaly prewarning information in a preset format to the portable electronic device 3.

In one of other embodiments of the present disclosure, after step W3, the diaper-based anomaly analysis and prewarning method includes the following steps:
V6, if the anomaly prewarning information needs to be sent, the monitoring device 1 sends a prewarning information sending request to the background server 5; and
V7, the background server 5 sends the anomaly prewarning information in the preset format to the portable electronic device 3 in response to the sending request.

In one of other embodiments of the present disclosure, the electronic tag component 6 in the diaper 2 is replaced with a second radio frequency electronic tag, the electronic device with the human perception unit shown in FIG. 12 and the monitoring device 1 shown in FIG. 7 can be the same electronic device, at the moment, the monitoring device 1 further includes the human perception unit in communication connection to the processor 10, and the diaper-based anomaly analysis and prewarning method includes the following steps:
the monitoring device 1 sends a radio frequency signal in real time or regularly based on the RFID card reader 15;
an electric small loop antenna and half-wave dipole antennae in the second radio frequency electronic tag send a second radio frequency signal when generating electromagnetic induction with the radio frequency signal;
the monitoring device 1 detects human information in a specific area based on the human perception unit in real time or regularly or after receiving a detection instruction sent by a user, analyzes the detected human information based on a third analysis algorithm so as to analyze whether the second radio frequency signal needs to be analyzed;
if the second radio frequency signal needs to be analyzed, the monitoring device 1 detects the second radio frequency signal in real time or regularly based on the RFID card reader 15, and analyzes a signal value of the detected second radio frequency signal based on a second analysis algorithm so as to analyze whether anomaly prewarning information needs to be sent; and
if the anomaly prewarning information needs to be sent, the monitoring device 1 directly sends anomaly prewarning information in a preset format to the portable electronic device 3, or if the anomaly prewarning information needs to be sent, the monitoring device 1 sends a prewarning information sending request to the background server 5, and the background server 5 sends the anomaly prewarning information in the preset format to the portable electronic device 3 in response to the sending request.

It should be noted that the contribution of the present disclosure to the prior art mainly lies in that: the overall technical solution breaks the inertial research and development thought of the skilled in the art continuing a research and develop path in a physical direction of the diaper itself, and successfully and non-obviously applies a research and development path and a technical solution which are environment-friendly, safe in use, convenient to operate, controllable in cost and accurate in anomaly prewarning in addition to the research and development path in the physical direction of the diaper itself. In above embodiments, detailed implementation solutions for specific technical features (such as the first analysis algorithm, the second analysis algorithm, and the third analysis algorithm) are exemplarily described, the exemplary description for these detailed implementation solutions is only based on the current industrial application conditions and technical conditions and is beneficial to industrial implementation, but does not mean that these specific technical features can only adopt the detailed implementation solutions in the exemplary description. According to an inventive concept proposed in the present disclosure, there is a possibility that the skilled in the art seek any other applicable detailed implementation solutions for the detailed implementation solutions.

In addition, an embodiment of the present disclosure further provides a nonvolatile computer-readable storage medium which can be any one or any combinations of a hard disk, a multimedia card, an SD card, a flash memory card, an SMC, a read-only memory (ROM), an erasable programmable read-only memory (EPROM), a portable compact disc-read-only memory (CD-ROM), a USB memory, etc. The nonvolatile computer-readable storage medium stores the diaper-based anomaly analysis and prewarning program 12 in the monitoring device 1 shown in FIG. 7, and the diaper-based anomaly analysis and prewarning program 12 can be called and executed by at least one processor to provide intelligent, environment-friendly and accurate diaper-based anomaly analysis and prewarning service for special crowds.

A specific software function implementation of the nonvolatile computer-readable storage medium of the present disclosure is approximately the same with the specific software implementation in the above-mentioned monitoring device 1 so as to be no longer repeated herein.

It should be noted that terms “includes”, “including” or any other variants thereof are intended to cover non-excludable inclusion, so that a process, apparatus, article or method including a series of elements not only includes those elements, but also includes other elements not listed clearly, or further includes inherent elements of the process, apparatus, article or method. In the case that there are no more limitations, an element defined by the word “including a . . . ” does not exclude other same elements further existing in the process, apparatus, article or method including the element

Serial numbers of the above-mentioned embodiments of the present disclosure are only for the purpose of description, but do not represent advantages or disadvantages of the embodiments. By means of the description for above implementations, it can be clearly known by the skilled in the art that the method in the above-mentioned embodiments can be implemented by means of software and an essential general-purpose hardware platform, of course, the method can also be implemented by means of hardware, however, the former is a better implementation in a plurality of cases. Based on such understanding, through the description for the above-mentioned implementations, it may be clearly known by the skilled in the art that all the implementations may be implemented by means of software and an essential universal hardware platform, of course, they may also be implemented by means of hardware. Based on such understanding, the essences of the technical solutions of the present disclosure or parts thereof making contributions to the prior art can be embodied in a form of a software product, and the computer software product is stored in a storage medium (such as an ROM/RAM, a diskette, and an optical disk) mentioned as above, and includes a plurality of instructions used to enable a terminal device (which can be a mobile phone, a computer, a server, or a network device, etc.) to perform the method in each of the embodiments of the present disclosure.

Above descriptions are only preferred embodiments of the present disclosure, and are not intended to hence limit the patent scope of the present disclosure. Any equivalent structure or flow transformations made according to the contents of the description and the accompanying drawings of the present disclosure are directly or indirectly applied to other related technical fields and also fall within the patent protection scope of the present disclosure in a similar way.

What is claimed is:

1. A diaper-based anomaly analysis method, the method being applicable to a diaper configured with an electronic tag component, and the electronic tag component comprising a first radio frequency electronic tag for detecting a first radio frequency signal representing that the diaper is located in a perceptible area and a second radio frequency electronic tag for detecting a second radio frequency signal representing the existence of a liquid, wherein the method comprises a step of analyzing anomaly prewarning and a step of sending anomaly prewarning information, wherein
the step of analyzing anomaly prewarning comprises:
sending a radio frequency signal in real time or regularly by a monitoring device so that the first radio frequency electronic tag of the electronic tag component sends the first radio frequency signal when generating electromagnetic induction with the radio frequency signal and the second radio frequency electronic tag sends the second radio frequency signal when generating electromagnetic induction with the radio frequency signal, detecting the first radio frequency signal in real time or regularly or after receiving a detection instruction sent by a user, analyzing a signal value of the detected first radio frequency signal based on a first analysis algorithm so as to analyze whether the second radio frequency signal needs to be analyzed, and if the second radio frequency signal needs to be analyzed, detecting the second radio frequency signal in real time or regularly, and analyzing a signal value of the detected second radio frequency signal based on a second analysis algorithm so as to analyze whether anomaly prewarning information needs to be sent; or
sending a radio frequency signal in real time or regularly by a monitoring device so that the first radio frequency electronic tag of the electronic tag component sends the first radio frequency signal when generating electromagnetic induction with the radio frequency signal and the second radio frequency electronic tag sends the second radio frequency signal when generating electromagnetic induction with the radio frequency signal, detecting the first radio frequency signal and the second radio frequency signal in real time or regularly or after receiving a detection instruction sent by a user, analyzing a signal value of the detected first radio frequency signal based on a first analysis algorithm so as to analyze whether the second radio frequency signal needs to be analyzed, and if the second radio frequency signal needs to be analyzed, analyzing a signal value of the detected second radio frequency signal based on a second analysis algorithm so as to analyze whether anomaly prewarning information needs to be sent; and
the step of sending anomaly prewarning information comprises:
if anomaly prewarning information needs to be sent, sending anomaly prewarning information in a preset format to a predetermined electronic device by a monitoring device; or
if anomaly prewarning information needs to be sent, sending a prewarning information sending request to a background server by a monitoring device so that the background server sends anomaly prewarning information in a preset format to a portable electronic device in response to the sending request.

2. The diaper-based anomaly analysis method of claim 1, wherein the first analysis algorithm comprises:
analyzing a signal value of a first radio frequency signal detected currently so as to analyze whether the signal value of the first radio frequency signal is greater than a first preset value;
if the signal value of the first radio frequency signal detected currently is greater than the first preset value, determining that the second radio frequency signal needs to be analyzed; and
if the signal value of the first radio frequency signal detected currently is smaller than or equal to the first preset value, determining that the second radio frequency signal does not need to be analyzed.

3. The diaper-based anomaly analysis method of claim 1, wherein the first analysis algorithm comprises:
analyzing a signal value of a first radio frequency signal detected currently so as to analyze whether the signal value of the first radio frequency signal is greater than a first preset value;
if the signal value of the first radio frequency signal detected currently is greater than the first preset value, analyzing signal values of a first preset number of first radio frequency signals detected continuously so as to analyze whether a variation amplitude value of the signal values of the first preset number of first radio frequency signals detected continuously is greater than a second preset value; and
if the variation amplitude value is smaller than or equal to the second preset value, determining that the second radio frequency signal needs to be analyzed, or if the variation amplitude value is greater than the second preset value, determining that the second radio frequency signal does not need to be analyzed.

4. The diaper-based anomaly analysis method of claim 1, wherein the second analysis algorithm comprises:
analyzing a signal value of a second radio frequency signal detected currently so as to analyze whether the signal value of the second radio frequency signal detected currently is greater than a third preset value;
if the signal value of the second radio frequency signal detected currently is greater than the third preset value, determining that the anomaly prewarning information does not need to be sent; and
if the signal value of the second radio frequency signal detected currently is smaller than or equal to the third preset value, determining that the anomaly prewarning information needs to be sent.

5. The diaper-based anomaly analysis method of claim 1, wherein the second analysis algorithm comprises:
analyzing a signal value of a second radio frequency signal detected currently so as to analyze whether the signal value of the second radio frequency signal detected currently is greater than a third preset value;
if the signal value of the second radio frequency signal detected currently is smaller than or equal to the third preset value, analyzing signal values of a second preset number of second radio frequency signals detected continuously so as to analyze whether there are second radio frequency signals of which signal values are greater than the third preset value in the second preset number of second radio frequency signals detected continuously; and
if there are the second radio frequency signals of which the signal values are greater than the third preset value, returning to perform the step of detecting the second radio frequency signal in real time or regularly and steps after the step, or if the signal values of all the second radio frequency signals are smaller than or equal to the third preset value, determining that the anomaly prewarning information needs to be sent.

6. The diaper-based anomaly analysis method of claim 1, wherein the second analysis algorithm comprises:
analyzing a signal value of a second radio frequency signal detected currently so as to analyze whether the signal value of the second radio frequency signal detected currently is greater than a third preset value;
if the signal value of the second radio frequency signal detected currently is smaller than or equal to the third preset value, analyzing signal values of a second preset number of second radio frequency signals detected continuously so as to analyze whether there are second radio frequency signals of which signal values are greater than the third preset value in the second preset number of second radio frequency signals detected continuously;
if there are the second radio frequency signals of which the signal values are greater than the third preset value, returning to perform the step of detecting the second radio frequency signal in real time or regularly and steps after the step;
if the signal values of all the second radio frequency signals are smaller than or equal to the third preset value, analyzing the signal values of the second preset number of second radio frequency signals detected continuously so as to analyze whether a variation amplitude value of the signal values of the second preset number of second radio frequency signals detected continuously is greater than a fourth preset value; and
if the variation amplitude value is smaller than or equal to the fourth preset value, returning to perform the step of detecting the second radio frequency signal in real time or regularly and steps after the step, or if the variation amplitude value is greater than the fourth preset value, determining that the anomaly prewarning information needs to be sent.

7. An electronic tag component being applicable to the diaper-based anomaly analysis method of claim 1, and the electronic tag component comprising a first radio frequency electronic tag for detecting a first radio frequency signal representing that the diaper is located in a perceptible area and a second radio frequency electronic tag for detecting a second radio frequency signal representing the existence of a liquid, wherein
the first radio frequency electronic tag comprises a first conductive substrate, and the first conductive substrate comprises a first substrate segment, a second substrate segment, and a transition substrate segment electrically connected to the first substrate segment and the second substrate segment;
the second radio frequency electronic tag comprises a first half-wave dipole antenna, a second half-wave dipole antenna, and a transition antenna segment electrically connected to the first half-wave dipole antenna and the second half-wave dipole antenna;
the first substrate segment is provided with a linear slot and a first tag chip disposed on one side of the slot, the first substrate segment and the second substrate segment are horizontally misaligned in a length direction, and the second substrate segment is located on an upper side of the first substrate segment in a width direction;
the transition antenna segment is provided with an electric small loop antenna, and the electric small loop antenna is provided with a second tag chip; and
the second substrate segment is located on an upper side of the first half-wave dipole antenna, and the second substrate segment and the first half-wave dipole antenna are parallel in the length direction.

8. The electronic tag component of claim 7, wherein the first half-wave dipole antenna, the second half-wave dipole antenna and the transition antenna segment are located in the same straight line in the length direction.

9. The electronic tag component of claim 7, wherein the first substrate segment and the first half-wave dipole antenna are located in the same straight line in the length direction.

10. The electronic tag component of claim 7, wherein a misalignment distance between the second substrate segment and the first substrate segment which are horizontally misaligned in the length direction is greater than the maximum width of the first half-wave dipole antenna in the width direction, and is smaller than two times of the maximum width of the first substrate segment in the width direction, or smaller than two times of the maximum width of the first half-wave dipole antenna in the width direction, or smaller than two times of a larger width value between the maximum width of the first substrate segment in the width direction and the maximum width of the first half-wave dipole antenna in the width direction.

11. A diaper applicable to the diaper-based anomaly analysis method of claim 1, and the diaper comprising a surface layer, an acquisition distribution layer, a water absorption layer, and a back sheet, wherein the diaper further comprises an electronic tag component fixedly mounted on the acquisition distribution layer and/or the back sheet and comprising a first radio frequency electronic tag for detecting a first radio frequency signal representing that the diaper is located in a perceptible area and a second radio frequency electronic tag for detecting a second radio frequency signal representing the existence of a liquid, wherein
the first radio frequency electronic tag comprises a first conductive substrate, and the first conductive substrate comprises a first substrate segment, a second substrate segment, and a transition substrate segment electrically connected to the first substrate segment and the second substrate segment;
the second radio frequency electronic tag comprises a first half-wave dipole antenna, a second half-wave dipole antenna, and a transition antenna segment electrically connected to the first half-wave dipole antenna and the second half-wave dipole antenna;
the first substrate segment is provided with a linear slot and a first tag chip disposed on one side of the slot, the first substrate segment and the second substrate segment are horizontally misaligned in a length direction, and the second substrate segment is located on an upper side of the first substrate segment in a width direction;
the transition antenna segment is provided with an electric small loop antenna, and the electric small loop antenna is provided with a second tag chip; and
the second substrate segment is located on an upper side of the first half-wave dipole antenna, and the second substrate segment and the first half-wave dipole antenna are parallel in the length direction.

12. The diaper of claim 11, wherein the first half-wave dipole antenna, the second half-wave dipole antenna and the transition antenna segment are located in the same straight line in the length direction.

13. The diaper of claim 11, wherein the first substrate segment and the first half-wave dipole antenna are located in the same straight line in the length direction.

14. The diaper of claim 11, wherein a misalignment distance between the second substrate segment and the first substrate segment are horizontally misaligned in the length direction is greater than the maximum width of the first half-wave dipole antenna in the width direction, and is smaller than two times of the maximum width of the first substrate segment in the width direction, or smaller than two times of the maximum width of the first half-wave dipole antenna in the width direction, or smaller than two times of a larger width value between the maximum width of the first substrate segment in the width direction and the maximum width of the first half-wave dipole antenna in the width direction.

15. A monitoring device, the monitoring device being matched with a diaper configured with an electronic tag component, and the electronic tag component comprising a first radio frequency electronic tag for detecting a first radio frequency signal representing that the diaper is located in a perceptible area and a second radio frequency electronic tag for detecting a second radio frequency signal representing the existence of a liquid, wherein the monitoring device comprises a communication unit, a memory, an RFID (Radio Frequency Identification) card reader, and a processor in communication connection to the communication unit, the memory and the RFID card reader, respectively, the memory stores a diaper-based anomaly analysis and prewarning program callable and executable for the processor, and when the diaper-based anomaly analysis and prewarning program is called and executed by the processor, a step of analyzing anomaly prewarning and a step of sending anomaly prewarning information are performed, wherein
the step of analyzing anomaly prewarning comprises:
sending a radio frequency signal in real time or regularly so that the first radio frequency electronic tag sends the first radio frequency signal when generating electromagnetic induction with the radio frequency signal and the second radio frequency electronic tag sends the second radio frequency signal when generating electromagnetic induction with the radio frequency signal, detecting the first radio frequency signal in real time or regularly or after receiving a detection instruction sent by a user, analyzing a signal value of the detected first radio frequency signal based on a first analysis algorithm so as to analyze whether the second radio frequency signal needs to be analyzed, and if the second radio frequency signal needs to be analyzed, detecting the second radio frequency signal in real time or regularly, and analyzing a signal value of the detected second radio frequency signal based on a second analysis algorithm so as to analyze whether anomaly prewarning information needs to be sent; or
sending a radio frequency signal in real time or regularly so that the first radio frequency electronic tag sends the first radio frequency signal when generating electromagnetic induction with the radio frequency signal and the second radio frequency electronic tag sends the second radio frequency signal when generating electromagnetic induction with the radio frequency signal, detecting the first radio frequency signal and the second radio frequency signal in real time or regularly or after receiving a detection instruction sent by a user, analyzing a signal value of the detected first radio frequency signal based on a first analysis algorithm so as to analyze whether the second radio frequency signal needs to be analyzed, and if the second radio frequency signal needs to be analyzed, analyzing a signal value of the detected second radio frequency signal based on a second analysis algorithm so as to analyze whether anomaly prewarning information needs to be sent; and
the step of sending anomaly prewarning information comprises:
if anomaly prewarning information needs to be sent, sending anomaly prewarning information in a preset format to a predetermined electronic device; or if anomaly prewarning information needs to be sent, sending a prewarning information sending request to a background server so that the background server sends anomaly prewarning information in a preset format to a portable electronic device in response to the sending request.

16. The monitoring device of claim 15, wherein the first analysis algorithm comprises:
analyzing a signal value of a first radio frequency signal detected currently so as to analyze whether the signal value of the first radio frequency signal is greater than a first preset value;
if the signal value of the first radio frequency signal detected currently is greater than the first preset value, determining that the second radio frequency signal needs to be analyzed; and
if the signal value of the first radio frequency signal detected currently is smaller than or equal to the first preset value, determining that the second radio frequency signal does not need to be analyzed.

17. The monitoring device of claim 15, wherein the first analysis algorithm comprises:
analyzing a signal value of a first radio frequency signal detected currently so as to analyze whether the signal value of the first radio frequency signal is greater than a first preset value;
if the signal value of the first radio frequency signal detected currently is greater than the first preset value, analyzing signal values of a first preset number of first radio frequency signals detected continuously so as to analyze whether a variation amplitude value of the signal values of the first preset number of first radio frequency signals detected continuously is greater than a second preset value; and
if the variation amplitude value is smaller than or equal to the second preset value, determining that the second radio frequency signal needs to be analyzed, or if the variation amplitude value is greater than the second preset value, determining that the second radio frequency signal does not need to be analyzed.

18. The monitoring device of claim 15, wherein the second analysis algorithm comprises:
analyzing a signal value of a second radio frequency signal detected currently so as to analyze whether the signal value of the second radio frequency signal detected currently is greater than a third preset value;
if the signal value of the second radio frequency signal detected currently is greater than the third preset value, determining that the anomaly prewarning information does not need to be sent; and
if the signal value of the second radio frequency signal detected currently is smaller than or equal to the third preset value, determining that the anomaly prewarning information needs to be sent.

19. The monitoring device of claim 15, wherein the second analysis algorithm comprises:
analyzing a signal value of a second radio frequency signal detected currently so as to analyze whether the signal value of the second radio frequency signal detected currently is greater than a third preset value;
if the signal value of the second radio frequency signal detected currently is smaller than or equal to the third preset value, analyzing signal values of a second preset number of second radio frequency signals detected continuously so as to analyze whether there are second radio frequency signals of which signal values are greater than the third preset value in the second preset number of second radio frequency signals detected continuously; and
if there are the second radio frequency signals of which the signal values are greater than the third preset value, returning to perform the step of detecting the second radio frequency signal in real time or regularly and steps after the step, or if the signal values of all the second radio frequency signals are smaller than or equal to the third preset value, determining that the anomaly prewarning information needs to be sent.

20. The monitoring device of claim 15, wherein the second analysis algorithm comprises:
analyzing a signal value of a second radio frequency signal detected currently so as to analyze whether the signal value of the second radio frequency signal detected currently is greater than a third preset value;
if the signal value of the second radio frequency signal detected currently is smaller than or equal to the third preset value, analyzing signal values of a second preset number of second radio frequency signals detected continuously so as to analyze whether there are second radio frequency signals of which signal values are greater than the third preset value in the second preset number of second radio frequency signals detected continuously;
if there are the second radio frequency signals of which the signal values are greater than the third preset value, returning to perform the step of detecting the second radio frequency signal in real time or regularly and steps after the step;
if the signal values of all the second radio frequency signals are smaller than or equal to the third preset value, analyzing the signal values of the second preset number of second radio frequency signals detected continuously so as to analyze whether a variation amplitude value of the signal values of the second preset number of second radio frequency signals detected continuously is greater than a fourth preset value; and
if the variation amplitude value is smaller than or equal to the fourth preset value, returning to perform the step of detecting the second radio frequency signal in real time or regularly and steps after the step, or if the variation amplitude value is greater than the fourth preset value, determining that the anomaly prewarning information needs to be sent.
